(19) United States
(12) Patent Application Publication
(10) Pub. No.: US 2023/0414343 A1
(43) Pub. Date: Dec. 28, 2023

(54) DMEK ENDOTHELIUM-IN DELIVERY DEVICE

(71) Applicant: Lions World Vision Institute, Inc., Tampa, FL (US)

(72) Inventors: Eric Abdullayev, Tarpon Springs, FL (US); Arthur C. Kurz, Safety Harbor, FL (US)

(21) Appl. No.: 18/340,142

(22) Filed: Jun. 23, 2023

Related U.S. Application Data
(63) Continuation-in-part of application No. 17/902,371, filed on Sep. 2, 2022.
(60) Provisional application No. 63/354,942, filed on Jun. 23, 2022.

(51) Int. Cl. A61F 2/14

(52) U.S. Cl. CPC A61F 2/142; A61F 2/148

(57) ABSTRACT

Delivery devices, carriers, and methods for endothelial keratoplasty surgical procedures are provided. The delivery devices include a flat chamber sized to accommodate thin ophthalmic tissue used in Descemet's Membrane Endothelial Keratoplasty (“DMEK”) procedures while the ophthalmic tissue is in a trifolded configuration with the endothelial cells facing inward. One end of the delivery devices is configured for insertion into a patient's eye to inject ophthalmic tissue. Carriers include a container and a cap configured to seal an opening of the container. The carrier is sized to accommodate a delivery device disposed within the container for storage and transport of ophthalmic tissue.

### CROSS-REFERENCE TO RELATED APPLICATIONS

This application claims priority to U.S. Provisional Application No. 63/354,942 filed Jun. 23, 2022. This application is also a continuation-in-part of U.S. Nonprovisional patent application Ser. No. 17/902,371 filed on Sep. 2, 2022, which itself claims priority from provisional application No. 63/240,144. Application Ser. No. 17/902,371 also claims priority from U.S. Nonprovisional patent application Ser. No. 16/727,217 filed on Dec. 26, 2019, which itself claims priority to U.S. Provisional Application No. 62/785,368 filed on Dec. 27, 2018 and U.S. Provisional Application No. 62/785,430 filed on Dec. 27, 2018. This application further claims priority to U.S. Provisional Application No. 63/354,942 filed on Jun. 23, 2022. The entire disclosures of the above applications are incorporated herein by reference.

### TECHNICAL FIELD AND BACKGROUND

The present technology relates to devices and methods for the storage and transport of donor ophthalmic tissue and introducing ophthalmic tissue into an anterior chamber of an eye. More particularly, the technology relates to fluid and ophthalmic injection delivery devices and methods of using the same for the storage, transportation, and transplantation of Descemet's membrane and endothelium into a recipient's cornea in correct anatomical orientation.

Ophthalmic tissue transplantation can be dramatically improved using endothelial keratoplasty (“EK”) surgical techniques. Such surgical techniques include Descemet's Membrane Endothelial Keratoplasty (“DMEK”) where a layer of the Descemet's membrane with endothelial cells can be removed from a donor cornea and transplanted into a patient recipient's eye. DMEK may provide improved post-operative visual outcomes, faster recovery times, and reduced rates of rejection compared to other endothelial keratoplasty procedures, such as Descemet stripping automated endothelial keratoplasty (“DSAEK”).

Despite the benefits of DMEK procedures, surgeons or patients commonly elect to perform other types of transplant procedures owing to the greater degree of difficulty in performing DMEK procedures. Much of this difficulty stems from the thinness and fragility of the grafts used in DMEK, which can be approximately 7 to 10 microns thick, as compared to grafts used in DSAEK procedures that are typically between 40 to 200 microns thick.

Delivery of ophthalmic tissue, including the fragile and thin layer of the Descemet's membrane from the donor cornea, can be a difficult process. The donor tissue must be carefully removed from the donor cornea, safely stored and transported in a sterile manner to the surgical site to mitigate the risk of damaging the tissue and then delivered into a donor's anterior chamber.

Due to inherent tissue properties, after removed from the donor cornea, a DMEK graft naturally scrolls tightly backwards on itself with the fragile endothelial cells facing outward (the “endo-out” position) and the stromal side facing inward. This increases the risk of damage to the endothelial tissue during transport as the endothelial cells contact the surface of the injection delivery device. Moreover, surgeons must insert the folded DMEK graft into the anterior chamber and unfold the DMEK graft so that the endothelial cells face inward toward the interior of the patient's eye. This unfolding process becomes significantly more challenging for thin DMEK grafts.

Ophthalmic tissue storage and transplant devices must allow surgeons to carefully deliver the tissue for transplant to the recipient patient without damaging the thin layer of endothelium cells that are important for restoring healthy vision. The corneal endothelium serves the important function of pumping fluid out of the corneal stroma to prevent the occurrence of edematous haze, which results in cloudy vision. Notably, the corneal endothelium does not regenerate following damage or loss of cells, so preventing damage to the endothelium during transplant is critical for successful transplant surgery.

To facilitate the transplantation, a tissue delivery device, such as an injector, can be preloaded with the ophthalmic tissue, and the delivery device is used to insert the ophthalmic tissue into the eye. DMEK procedures require that an incision be made in the patient's eye prior to introducing the donor graft into the patient's anterior chamber via the delivery device. By providing an endo-in delivery device preloaded with the ophthalmic tissue, the time investment in the transplantation procedure can be minimized.

Given the challenges with performing DMEK procedures, it is an object of the present invention to provide delivery devices and methods that allow for safe, reliable storage, transport, and transplant of thin corneal tissue grafts into a patient's anterior chamber in the correct anatomical position while minimizing damage to the sensitive endothelial cells. The delivery devices transplant tissue into a patient's eye using fluid injection techniques that eliminate the need to physically contact the fragile corneal tissue, thereby mitigating damage.

The delivery devices and methods disclosed herein are an improvement over conventional devices because the present delivery devices inject the graft into anterior chamber with endothelial cells facing inward. Injecting a DMEK graft into an anterior chamber of a patient's eye with the endothelial cells facing inward is less challenging and less traumatic than conventional procedures because the DMEK graft naturally unfolds into the correct anatomical configuration with the endothelium cell layer facing inward to the interior of the patient's eye.

### SUMMARY

The present technology includes articles of manufacture, systems, and processes that relate to storage, transport, and introducing ophthalmic tissue, including Descemet's membrane and endothelium cell layers into a recipient patient's eye for various restorative procedures, including DSAEK, DMEK, and PDEK. The description and specific examples are intended for purposes of illustration only and are not intended to limit the scope of the present disclosure.

A first embodiment of a delivery apparatus for performing endothelial keratoplasty includes a first conduit having a beveled first end with a first opening, a second conduit having a second end with a second opening, and a chamber that extends between the first conduit and the second conduit along a first axis. The chamber has an interior thickness that extends along a second axis perpendicular to the first axis between interior surfaces, and the interior thickness is between 0.4 millimeters and 0.8 millimeters. The chamber has an interior width that extends along a third axis between interior surfaces where the width is perpendicular to both the first axis and the second axis, and the interior width is between 2 millimeters and 4 millimeters.

The endothelium delivery apparatus the first conduit can have an inner diameter between 1.1 millimeters and 2.2 millimeters, and the second conduit can be formed with an inner diameter between 1.1 millimeters and 2.2 millimeters. The delivery apparatus can be connected to a pressure-actuated valve. The pressure-actuated valve itself includes a first portion configured for coupling to a syringe as well as a valve main body, a deformable stopper disposed in the main body, and a second portion in fluid communication with the injector second end.

In another embodiment, the delivery apparatus can include an elongated resilient member having a channel extending from a first aperture to a second aperture. The resilient member is coupled to the second portion of the pressure-actuated valve by extending the second portion partially through the resilient member first aperture. The resilient member is coupled to the delivery apparatus second end by extending the second end partially through the resilient member second aperture. The delivery apparatus also includes a syringe filled with a balanced salt solution coupled to the first portion of the pressure actuated valve.

The delivery apparatus further includes a bulb disposed on an outer surface of the second conduit. The delivery apparatus has features that include a chamber for housing an ophthalmic tissue graft that has an endothelium cell layer and a stromal layer. The ophthalmic tissue graft is in a folded configuration with the endothelium layer facing inward and the stromal layer facing outward to contact a chamber inner surface, and the ophthalmic tissue graft endothelium layer is in contact with a corneal storage medium.

In another embodiment, a delivery apparatus for performing endothelial keratoplasty includes a first conduit having a beveled first end with a first opening, a second conduit having a second end with a second opening, and a chamber that extends between the first conduit and the second conduit. The chamber is configured with a flattened, hollow body that is wider than the first conduit and wider than the second conduit. The chamber is sized to accommodate cornea tissue with an endothelial cell layer on a first side while the cornea tissue is in a trifolded configuration with the endothelial cell layer facing inward away from an interior surface of the chamber. The second opening of the delivery apparatus is coupled to, and in fluid motion with, a fluid manipulation device.

In another embodiment, the delivery apparatus includes a delivery device formed with a container that has an opening and a cap configured to seal the opening of the container. At least a portion of the delivery device is disposed within the container. The delivery and the container are each at least partially filled with a corneal storage medium. In another feature, the delivery apparatus has a beveled first end that includes a leading portion and a trailing portion. The leading portion is made with a cutting surface for cutting and penetrating eye tissue, and the trailing portion comprises a non-cutting surface.

The delivery apparatus can also include a pressure-actuated valve coupled to the second end of the delivery apparatus. The delivery apparatus can have a syringe coupled to the pressure-actuated value. The pressure-actuated value has a first portion configured for coupling to a syringe, a valve main body, a deformable stopper disposed in the main body, and a second portion coupled to the second end of the second conduit. The pressure actuated value is coupled to the second end through a resilient member, and the resilient member includes a channel extending from a first aperture to a second aperture. The resilient member is coupled to the second portion of the pressure-actuated valve by extending the second portion partially through the resilient member first aperture, and the resilient member is coupled to the second end by extending the second end partially through the resilient member second aperture.

In one aspect of the invention, a method for storing an ophthalmic tissue graft with a layer of endothelial cells facing inward includes the steps of providing a delivery apparatus and loading the chamber with an ophthalmic tissue graft in a tri-folded configuration. The layer of endothelial cells is folded inward, and the layer of endothelium cells contacts the corneal storage medium. The ophthalmic tissue graft is oriented relative to the orientation of the beveled first end using the bevel-up, endo-down technique where the endothelium layer faces away from a sloping portion of the first end. The delivery device and carrier are transported to a site for performing endothelial keratoplasty.

Also disclosed is a method for performing endothelial keratoplasty that includes the steps of providing a delivery apparatus, loading the delivery apparatus with a corneal storage medium, and loading the delivery apparatus chamber with an ophthalmic tissue graft. The ophthalmic tissue graft has an endothelium layer and a stromal layer opposite the endothelium layer. The ophthalmic tissue graft is folded with the endothelium layer facing inward and the stromal layer facing outward to contact the chamber interior surface. The ophthalmic tissue graft is oriented within the chamber using the bevel-up, endo-down technique. The syringe is coupled to the first portion of the pressure-actuated valve, and the syringe is configured with a barrel containing a fluid, and a plunger partially housed within the barrel such that depressing the plunger applies pressure to the fluid.

The method also includes the step of creating an incision in the eye of a patient that is smaller than the outer diameter of the first conduit of the delivery apparatus. The steps also include inserting the beveled first end of the delivery apparatus into the eye of a patient through the incision where the beveled first end occupies substantially the entire incision. The bevel faces upward in a direction away from the interior of the patient's eye, and the syringe plunger is depressed to dispense the ophthalmic tissue graft from the chamber through the beveled first end and into the eye of the patient with the endothelial cell layer facing inward to the interior of the patient's eye. Depressing the plunger causes the fluid to flow from the barrel, through the pressure actuated valve and the delivery device, and into the patient's eye to exert a positive pressure within the eye. The fluid can be a balanced salt solution.

### BRIEF DESCRIPTION OF THE FIGURES

Features, aspects, and advantages of the present invention are better understood when the following detailed description of the invention is read with reference to the accompanying figures, in which:

FIG. 1 shows a top view of an embodiment of a DMEK endo-in delivery device (shown with loaded DMEK graft in the flat, chamber portion) for performing surgical procedures introducing ophthalmic tissue grafts into a patient's eye through injection.

FIG. 2 shows a side view of an embodiment of an endo-in delivery device for performing surgical procedures introducing ophthalmic tissue grafts into an eye.

FIG. 3A is a top view of an embodiment of the endo-in delivery device showing the device measurements.

FIG. 3B is a side view of an embodiment of the endo-in delivery device showing the device measurements.

FIG. 4 illustrates use of an endo-in delivery device for performing surgical procedures injecting an ophthalmic tissue grafts into an eye with the endothelium side down and stromal side up where the graft is stored in the flattened central chamber and exits the device through an injection end.

FIG. 5 illustrates the steps for using the endo-in delivery device for performing surgical procedures introducing ophthalmic tissue grafts into an eye.

FIG. 6 shows an exploded view of an endo-in delivery device, resilient member, and valve for performing surgical procedures introducing ophthalmic tissue grafts into an eye.

FIG. 7 shows an assembled view of an endo-in delivery device, resilient member, valve, and syringe for performing surgical procedures introducing ophthalmic tissue grafts into an eye.

FIG. 8 shows an assembled view of a carrier for an endo-in delivery device for performing surgical procedures introducing ophthalmic tissue grafts into an eye.

### DETAILED DESCRIPTION

The present invention will now be described more fully with reference to the accompanying pictures in which example embodiments of the invention are shown. However, the invention may be embodied in many different forms and should not be construed as limited to the representative embodiments set forth herein. The example embodiments are provided so that this disclosure will be both thorough and complete and will fully convey the scope of the invention to enable one of ordinary skill in the art to make, use, and practice the invention.

Relative terms such as lower or bottom; upper or top; upward, outward, or downward; forward or backward; and vertical or horizontal may be used herein to describe one element's relationship to another element illustrated in the figures. It will be understood that relative terms are intended to encompass different orientations in addition to the orientation depicted in the drawings. By way of example, if a component in the drawings is turned over, elements described as being on the “bottom” of the other elements would then be oriented on “top” of the other elements. Relative terminology, such as “substantially” or “about,” describe the specified materials, steps, parameters, or ranges as well as those that do not materially affect the basic and novel characteristics of the claimed inventions as whole. Regarding methods disclosed, the order of the steps presented is exemplary in nature, and thus, the order of the steps can be different in various embodiments. “A” and “an” as used herein indicate “at least one” of the item is present; a plurality of such items may be present, when possible.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

### Technology Applications and Conventional Techniques

The present technology improves administration of ophthalmic tissue into an eye using endothelial keratoplasty surgical techniques, including specifically Descemet's membrane endothelial keratoplasty. For these surgical techniques, at least a portion of the Descemet's membrane with endothelial cells is removed from a donor cornea and transplanted into a patient/recipient's eye. Once removed from a donor, the ophthalmic tissue can be loaded into an injector delivery device and housed within a delivery device chamber for storage and transport to the surgical site. Conventional delivery devices used for DMEK procedures can include, for example, a Weiss Ophthalmic Cannula—made by STRAIKO®—with a bulb-shaped chamber.

Within the chamber of conventional delivery devices, the thin tissue used for DMEK procedures naturally scrolls tightly on itself in an “endo-out” configuration where the delicate endothelial cells are facing outward. In the endo-out configuration, the endothelial cells are potentially exposed to damaging physical contact with the chamber interior sidewalls or during transplant surgery. To transplant the ophthalmic tissue, an incision is created in the patient's eye, and the ophthalmic tissue is inserted into the front portion (e.g., anterior chamber) of the eye using an injector delivery device.

One of the biggest challenges in performing a DMEK procedure is flattening out or unfolding the ophthalmic tissue graft so the endothelial cells will be positioned inward toward the interior of the patient's eye and the stromal side of the ophthalmic tissue graft can adhere to the posterior surface of the recipient's cornea without causing damage to the delicate endothelial cells. At present, there are limited options to open the scrolled layer of endothelial cells. The options for unscrolling the ophthalmic tissue include repeatedly tapping on the outer cornea, injecting air or fluid inside the eye, or manipulating the tissue further with surgical instrumentation. Such techniques are time consuming and risk damaging the delicate endothelial cells as the tissue is floated around in the anterior chamber of the eye. The corneal endothelium tissue is critical for clear vision, but the tissue does not regenerate following cell damage or loss. Consequently, avoiding harm to the corneal endothelium tissue is an important factor for successful surgery.

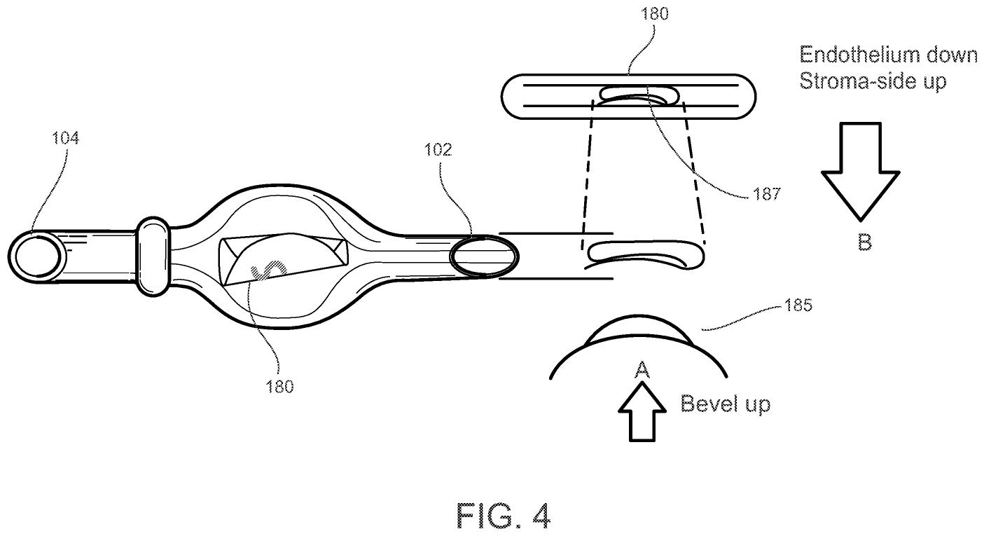

The novel “endothelium-in” delivery devices and techniques disclosed herein address the drawbacks of conventional devices and DMEK surgical techniques in part by retaining the tissue within a chamber in a trifolded configuration, depicted in FIG. 4, where the delicate endothelial cells are directed inward—i.e., an “endo-in” configuration. The endo-in configuration better protects the endothelial cells from damage during storage and transport because the cells are not exposed.

After removal from a donor eye, the ophthalmic tissue is trifolded into the endo-in configuration and loaded into the endo-in delivery device using, for example, a special loading forceps. The endo-in delivery device includes a chamber with a size and shape that does not allow the ophthalmic tissue to scroll into the natural endo-out position. During a DMEK procedure, the ophthalmic tissue is dispensed from the present endo-in delivery device into the anterior chamber the patient's eye while the tissue is still in the endo-in configuration, which is not a feature that can be currently achieved using existing delivery devices.

Once delivered into the anterior chamber, the thin ophthalmic tissue starts unfolding by naturally scrolling from an endo-in configuration to an endo-out configuration. Also, fluid injected from delivery device exerts fluid pressure that facilitates the ophthalmic tissue graft unfolding from the endo-in to the endo-out configuration. During the process of unfolding from an endo-in position, the ophthalmic tissue becomes properly seated within the patient's eye before reaching the endo-out configuration. In this manner, the ophthalmic tissue is transplanted without the need to touch, tap, or otherwise physically manipulate the tissue, which mitigates against the risk of tissue damage. Thus, the delivery devices disclosed herein provide significant advantages over existing devices and are particularly well suited for carrying out DMEK procedures that utilize thin tissue membranes that are otherwise difficult to manipulate.

### Delivery Device Embodiments Adapted for DMEK Procedures

An embodiment of an endo-in delivery device 100 is shown in the attached Figures and is particularly adapted for use in DMEK procedures to minimize ophthalmic tissue damage and to promote patient health and safety. Skilled artisans will appreciate that the below-described devices and techniques can be adapted for use in endothelial keratoplasty surgical procedures generally and PDEK procedures more specifically. However, the devices and techniques have particular advantages when applied to DMEK surgical techniques.

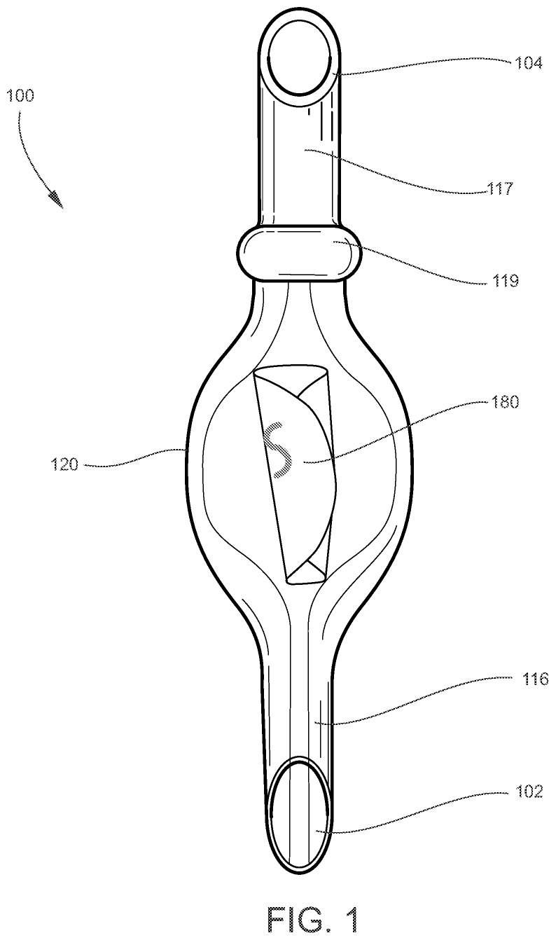

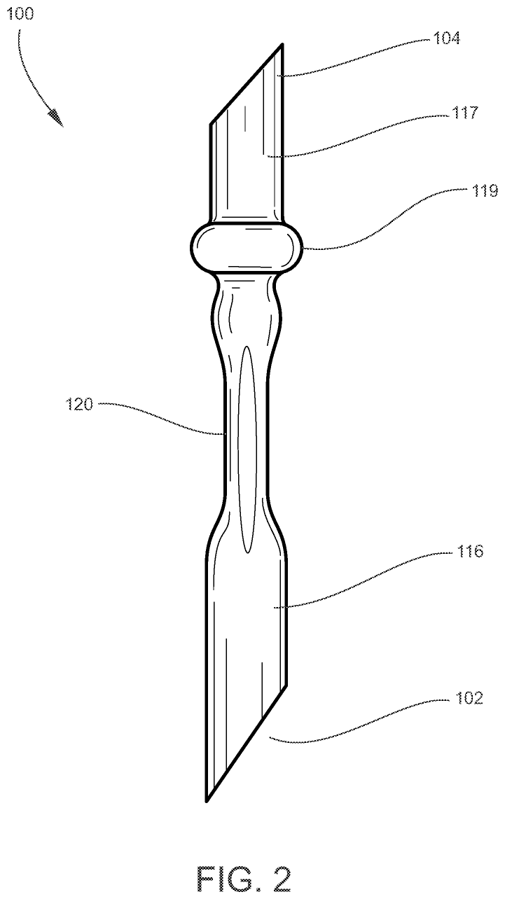

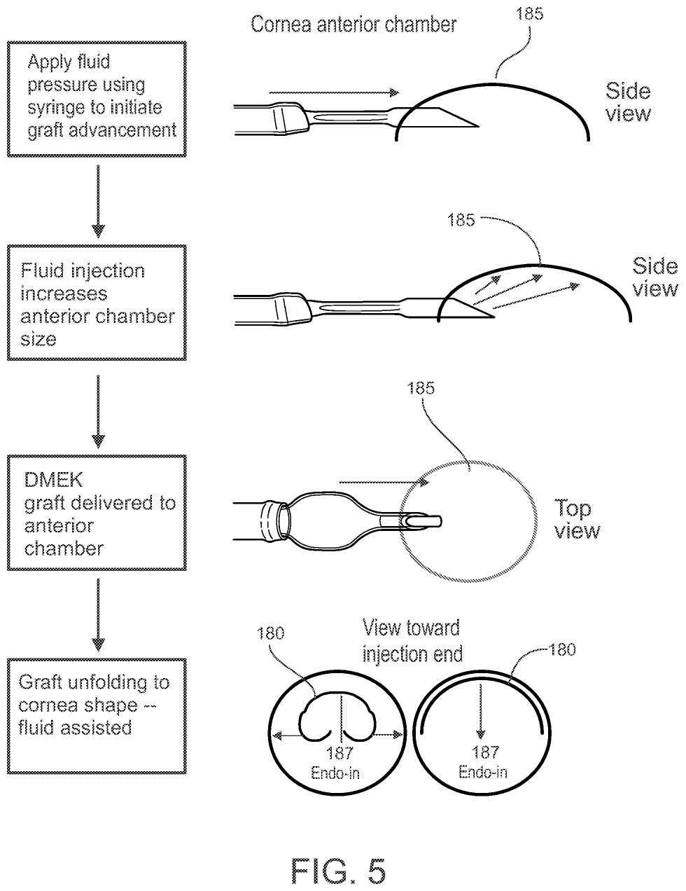

The endo-in delivery device 100 shown in FIGS. 1-4 is formed as an elongated hollow body that defines a first conduit 116, a flattened-shaped chamber 120 that houses ophthalmic tissue 180 in a trifolded configuration, and a second conduit 117. The endo-in delivery device 100 can be cylindrical with first end or “injection end” 102 with a first opening, a second end or “coupling end” 104 with a second opening, and a bulb 119. The bulb 119 is disposed between flat chamber 120 and coupling end 104. The bulb 119 serves to engage or define a coupling with an elastic resilient member 225 (FIG. 5) or a fluid manipulation device, such as a syringe having a barrel and plunger.

The injection end 102 is beveled to minimize damage to the incision during insertion of the endo-in delivery device 100 into the anterior chamber. That is, the injection end 102 can be configured so that the narrow leading portion enters the incision first without damaging the incision, and the beveled trailing portion follows without tearing or otherwise damaging the tissue as the endo-in delivery device 100 is inserted into the anterior chamber. In this manner, the wound profile of the incision is minimized, where the remainder of the beveled opening can push through the incision without having to increase the size of the incision opening. The coupling end 104 is beveled. When loading ophthalmic tissue 180 into the endo-in delivery device 100, the ophthalmic tissue is loaded through the coupling end 104 and travels through the second conduit 117 before being housed in the flattened shaped chamber 120. During loading, the ophthalmic tissue 180 is sucked into the coupling end 104 using negative pressure exerted by a syringe connected to the opposite injection end 102 of the device.

A chamber 120 extends between the conduits 116 & 117 where the chamber 120 and the conduits 116 & 117 extend along a first axis. The chamber 120 has a size and shape that is configured to house ophthalmic tissue 180 in the endo-in configuration such that the tissue 180 does not scroll back into the endo-out configuration. In particular, the chamber 120 is wide across a second axis that is transverse to the first axis, the chamber 120 has flattened parallel, sidewalls, as compared to conventional delivery devices that have a bulb or tapered form. The chamber 120 is also narrow along a third axis corresponding to the chamber 120 thickness where the third axis is transverse to both the first and second axes.

The chamber 120 and conduit 116 are also formed with sufficient interior dimensions to hold both ophthalmic tissue and a corneal storage media that provides irrigation. That is, the chamber 120 is sized to avoid the circumstance where the ophthalmic tissue 180 becomes folded or constrained in a manner that does not allow the corneal storage medium to contact the ophthalmic tissue 180, which could damage the tissue 180. The DMEK tissue graft can have a single layer thickness of approximately 10 microns before it is placed in a tri-fold configuration that is three-layers thick. The chamber 120 must, therefore, be larger than three times the thickness of the DMEK tissue graft but small enough to not permit the DMEK tissue graft to unfold from the endo-in configuration when the tissue graft is stored in the chamber.

The chamber 120 defined by the interior conduit is sized to allow the ophthalmic tissue 180 to be folded in the endo-in configuration as shown in FIG. 4 where the endothelium layer 187 is folded inward and does not contact the inner surface of the chamber 180. This configuration does not change during storage and transport and protects the delicate endothelium layer 187 during storage, transport, and transplant of the ophthalmic tissue 180. The orientation of the ophthalmic tissue folds relative to the orientation of the bevel in the injection end 102 of the endo-in delivery device 100 can play a role in successful transplant by facilitating the proper unfolding of the ophthalmic tissue after it is injected into the anterior chamber. The endo-in delivery device 100 shown in the attached figures allows the ophthalmic tissue folds to maintain their orientation during storage and transport, thereby enhancing the chances of a successful transplant surgery.

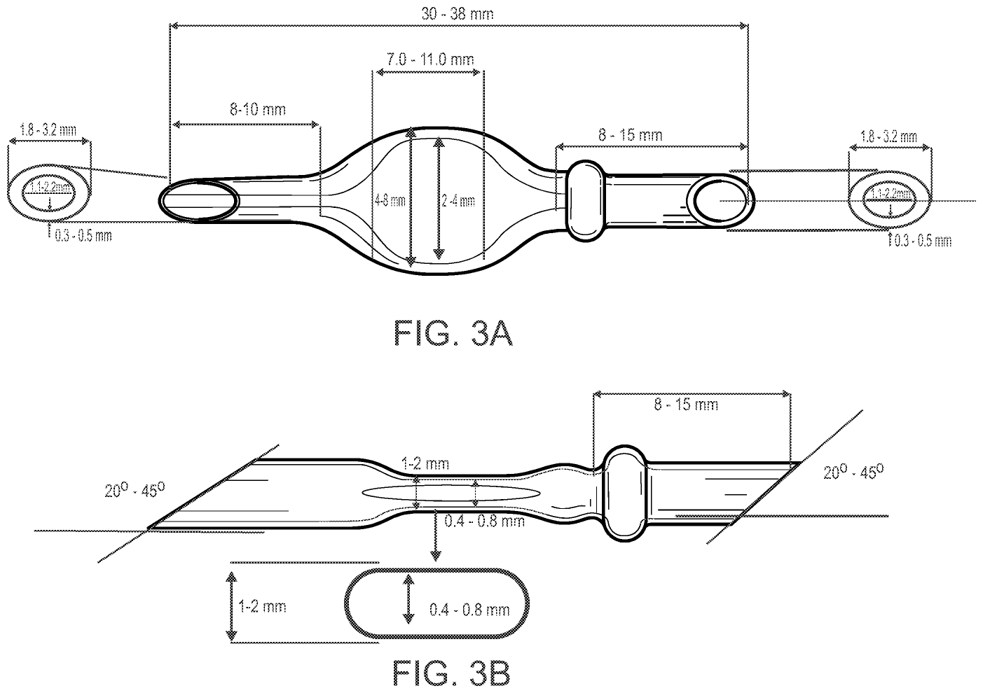

In one example embodiment illustrated in FIGS. 3A and 3B, the first and second conduits 116 & 117 have an inner diameter between 1.1 millimeters and 2.2 millimeters. The second conduit can also have an inner diameter between 1.1 millimeters and 2.2 millimeters. The injection end and the coupling end each have a bevel outer diameter that is between 1.8 millimeters and 3.2 millimeters and a sloping edge that is between 20 degrees and 45 degrees. The first conduit can have a length that is between 8 millimeters and 10.0 millimeters. The second conduit can have a length that is between 8.0 millimeters and 15.0 millimeters.

The hollow, flattened-shaped chamber 120 has an interior surface that defines a conduit where the conduit has a width between 4 millimeters and 8 millimeters between ends of the interior surface. The chamber has an exterior width between 4 millimeters and 8 millimeters that extends between the outer, exterior surfaces of the chamber. The chamber has a length that extends between the first conduit and the second conduit where the length is between 7 millimeters and 10 millimeters.

The hollow chamber has an interior thickness between four-tenths and eight-tenths (0.4 to 0.8) millimeters where the interior thickness extends between opposite ends of the interior sidewall. The hollow chamber also has an exterior thickness extends between outer, exterior sidewalls of 1 millimeter to 2 millimeters.

With reference to FIG. 4, during a DMEK surgical procedure, the beveled surface of the endo-in delivery device injection end 102 is pointed upward in the “A” direction while the endo-in delivery device 100 penetrates a patient's cornea 185. This allows the ophthalmic tissue 180 to be injected with the endothelium layer 187 pointed downward in the “B” direction. This technique is referred to as “bevel-up, endo-down” and allows the ophthalmic tissue 180 to naturally unfold and take the shape of a patient's cornea 185 after the tissue 180 is dispensed into the anterior chamber of the cornea 185.

The coupling end 104 of the endo-in delivery device 100 can optionally be formed with the same diameter or with a larger diameter than the injection end 102. For embodiments having a coupling end 104 with a larger diameter than the injection end 102, following extraction of ophthalmic tissue from a donor, the tissue is placed into a symmetrical trifold configuration using forceps and loaded into the endo-in delivery device 100 through the larger diameter coupling end 104. Both the injection end 102 and the coupling end 104 are formed with a bevel, and either end could be used during a surgical procedure to inject ophthalmic tissue into a patient's eye through an incision.

The endo-in delivery device 100 can be made from a glass material, such as 7740 Pyrex® borosilicate glass. In other embodiments, the endo-in delivery device 100 can be made from a plastic material that is less susceptible to breaking and less expensive to manufacture using blow molding, injection molding, or extrusion. In yet another embodiment, the beveled surface of the injection end 102 or coupling end 104 can be made from a metal material suitable for facilitating penetration of ophthalmic tissue.

The endo-in delivery device 100 can also be used in various methods of transporting ophthalmic tissue. Such methods can include where the ophthalmic tissue is obtained from a donor. A carrier as described herein is provided and the ophthalmic tissue is disposed within the endo-in delivery device 100. Corneal storage medium is provided within the container, where the corneal storage medium contacts the ophthalmic tissue when the ophthalmic tissue is positioned within the endo-in delivery device 100. The injector carrier, including the endo-in delivery device 100 with the ophthalmic tissue disposed therein, is then transported to a site for the endothelial keratoplasty, for example.

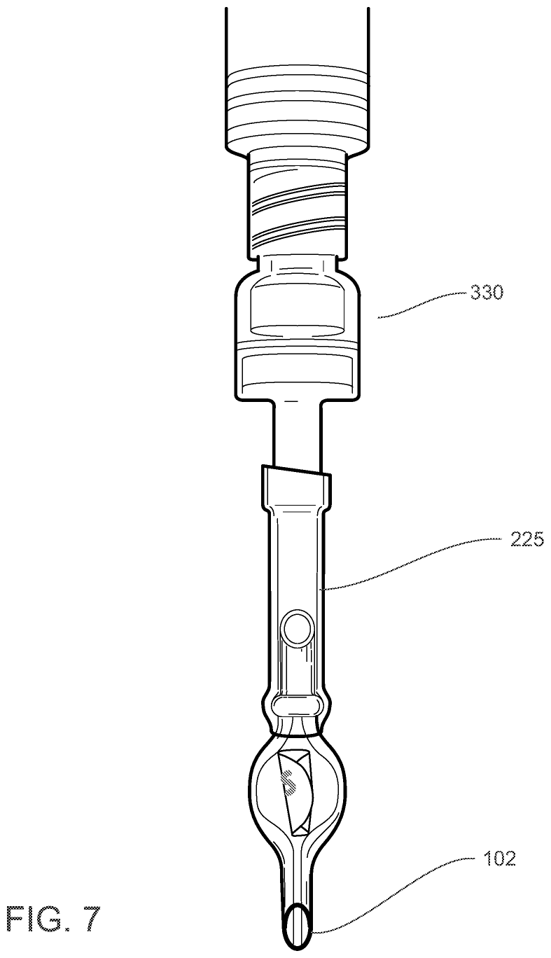

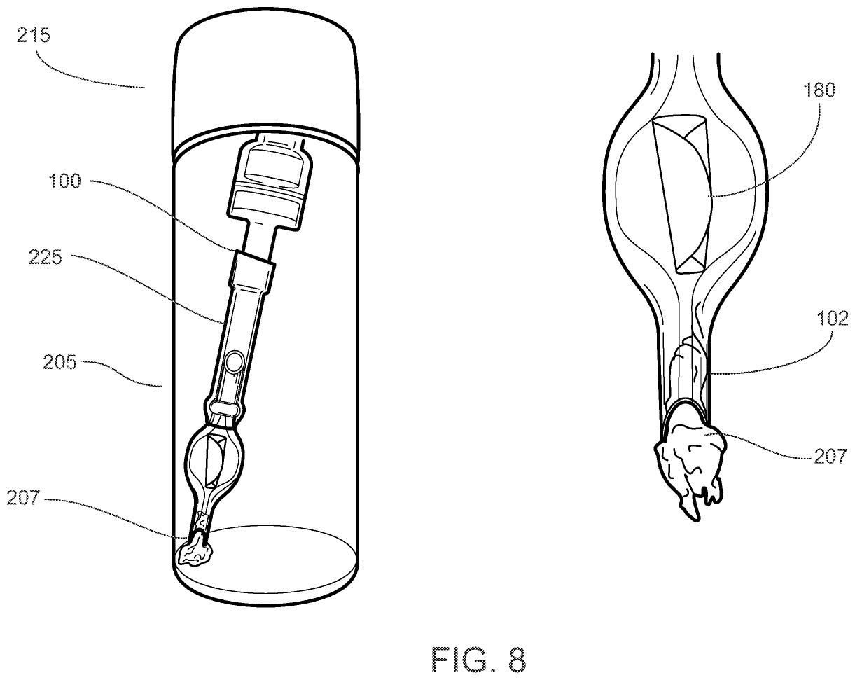

Turning to FIGS. 7-8, the endo-in delivery device 100 is shown with other components that facilitate storage, transportation, and transplant of ophthalmic tissue. The coupling end 104 of the endo-in delivery device 100 can be coupled to a first aperture at a first end 230 of a flexible, elongated resilient member 225. The resilient member 225 second aperture at a second end 235 is in turn coupled to the stem portion 336 of a pressure-activated Luer-lock valve 330. The resilient member 225 is hollow and defines a channel that places the endo-in delivery device second conduit 117 in fluid communication with the Luer-lock valve 330. The resilient member 225 is sized such that it fits over the bulb 119, coupling end 104, and stem portion 336 and compresses the bulb 119, coupling end 104, and stem portion 336 to create a seal that does not allow air or fluid to escape.

To store and transport the ophthalmic tissue, the endo-in delivery device 100 can be used in conjunction with an injector carrier 200 as shown in FIG. 8. The injector carrier 200 includes a container 205 having an opening (not shown) and a cap 215 configured to seal the opening 210 of the container 205. The container 205 of the injector carrier 200 holds an amount of corneal storage medium sufficient to contact ophthalmic tissue in the endo-in delivery device 100 when the endo-in delivery device 100 is disposed within the container 205 while the cap 215 seals the opening 210 of the container 205. A sponge 207 is inserted into the injection end 102 of the endo-in delivery device 100 while stored in the container 205 to prevent the ophthalmic tissue from inadvertently escaping the endo-in delivery device 100 prior to surgery.

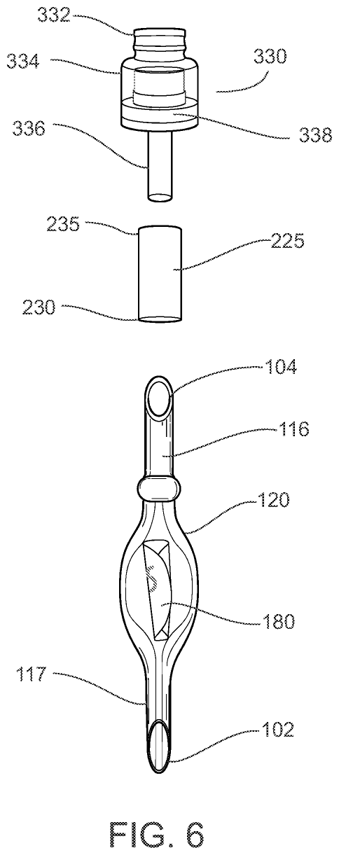

Turning again to FIGS. 6-7, the Luer-lock valve 330 includes a threaded feed portion 332, a main body 334, and a stem portion 336. The threaded feed portion 332 and the stem portion 336 both include an opening and a passage that are in fluid communication with the interior of the main body 334. The main body 334 houses a stopper 338 that can be made of silicone or another resilient, compressible material that deforms under pressure but returns to its original form when the pressure is removed. Absent applied pressure, the stopper 338 occupies the entire volume of the interior of the valve main body 334 so that fluid cannot pass from the passage of the threaded feed portion 332 to the passage of the stem portion 336. When pressure is applied to the stopper 338, the stopper 338 deforms and allows fluid to pass from the passage of the threaded feed portion 332 to the passage of the stem portion 336. When the pressure is removed, the stopper 338 returns to its prior shape, thereby cutting off fluid flow.

Those of skill in the art will appreciate that use of a resilient member 225 is not intended to be limiting, and other suitable components and configurations may be used to place a valve 330 in fluid communication with the endo-in delivery device 100. For example, the endo-in delivery device 100 may be made of a plastic material with a coupling end 104 opening sized to fit over the stem portion 336 of the Luer-lock valve 330. Alternatively, the coupling end 104 and stem portion 336 may be threaded so that the two components can be screwed together. Additionally, other types of valves may be used instead of the pressure actuated Luer-lock valve 330 to place the syringe 350 in fluid communication with the endo-in delivery device 100. Other embodiments may utilize a spring-tensioned valve or a butterfly-type valve where applied fluid pressure cause a disk or other stopper to move and allow fluid flow.

With respect to the embodiments shown in the attached figures, the Luer-lock valve 330 facilitates DMEK surgical procedures performed using the endo-in delivery device 100 described above. Prior to surgery, the threaded feed portion 332 of the Luer-lock valve 330 is connection to a syringe 350 having a barrel and a plunger, as depicted in FIG. 7. The syringe 350 is filled with a balanced salt solution suitable for injection into a patient's anterior chamber. The stem portion 336 of the Luer-lock valve 330 is coupled to the second portion 235 of the resilient member 225, and the first portion 230 of the resilient member 225 is connected to the coupling end 104 of the endo-in delivery device 100. The endo-in delivery device conduit 116 & 117 and chamber 120 house the ophthalmic tissue to be transplanted and is filled with corneal storage medium, such as Optisol-GS to facilitate sterile preservation of the tissue. With the components connected in this manner, the barrel of the syringe 350 is placed in fluid communication with the endo-in delivery device second conduit 117 through the resilient member 225 and the Luer-lock valve 330.

During surgery, the injection end 102 of the endo-in delivery device 100 is inserted through a pre-cut incision in a patient's cornea, and the injection end 102 is placed inside the anterior chamber with the bevel-side facing “up,” as shown in FIG. 5. Next, the ophthalmic tissue is injected into the anterior chamber by actuating the syringe plunger. When the plunger is depressed, the balanced salt solution exerts a positive pressure on the stopper 338. The stopper 338 deforms in response to the positive pressure and allows the balanced salt solution to pass from the syringe 350 barrel through the Luer-lock valve 330 and resilient member 225 to the endo-in delivery device conduit 116 & 117 and chamber 120. The balanced salt solution then exerts a positive pressure within the conduit 116 & 117 and chamber 120 that expels the ophthalmic tissue from the injection end 102 of the endo-in delivery device 100 into anterior chamber without the need to touch or mechanically contact the ophthalmic tissue. The ophthalmic tissue is expelled from the endo-in delivery device 100 along with balanced salt solution that flows into the anterior chamber and applies pressure to the ophthalmic tissue to facilitate the tissue unfolding so that the tissue takes the shape of the patient's cornea. The balanced salt solution injected into the anterior chamber also maintains ocular pressure and fluid levels within the anterior chamber.

### Working Examples of DMEK Surgical Procedures

The following examples were used to evaluate endothelial cell viability of prepared DMEK grafts of ophthalmic tissue in conjunction with use of the endo-in delivery device 100. All grafts of ophthalmic tissue were pre-loaded into an endo-in delivery device 100 for use in DMEK procedures. FIG. 5 depicts insertion of the injection end 102 of the first conduit 116 into the anterior chamber, wherein the beveled surface penetrates into the anterior chamber. The ophthalmic tissue is then dispensed from the first conduit 116 of the endo-in delivery device 100 through the first conduit 116 of the endo-in delivery device 100 into the anterior chamber.

A cut is made by the cutting surface. A mark is visible where the non-cutting surface pushes through into the anterior chamber, but does not cut the outer surface, as the entire opening at the injection end 102 of the first conduit 116 is inserted into the anterior chamber. In certain embodiments, the present technology provides various methods of using the injector carrier. These include ways of loading a graft of ophthalmic tissue into the endo-in delivery device, ways of assembling the injector carrier, and ways of administering the graft or ophthalmic tissue using the injector carrier (e.g., performing keratoplasty). The following exemplary methods include a series of steps where it will be evident to one skilled in the art that the order of certain steps can be different in various embodiments while the order of other certain steps cannot be changed relative to each other. Similarly, additional steps can be included in the various embodiments of the present technology and certain steps may be omitted in certain embodiments of the present technology.

Various embodiments of an endo-in delivery device and an injector carrier can be used as follows to load ophthalmic tissue (e.g., a prepared graft) as follows.

### Loading a Prepared Graft

1. Use universal scissors to cut a small (e.g., approximate ½ inch) piece of suction tubing to fit the injector.

2. Connect one side of the suction tubing to an end of the endo-in delivery device and the other side to a syringe.

3. Depress the plunger of the syringe to transfer media to the endo-in delivery device from the syringe, ensuring no air bubbles are present.

4. Place an end of the endo-in delivery device next to the prepared graft of ophthalmic tissue and use suction from the syringe to move the graft into the endo-in delivery device, until the graft is in a flattened-shaped central portion of the endo-in delivery device.

5. Gently disconnect the syringe from the tubing at the end of delivery device, making sure that the coupling end of the endo-in delivery device stays in the corneal storage medium during disconnection.

6. Reconnect syringe to the tubing at the coupling end of endo-in delivery device, making sure that the end of the endo-in delivery device remains submerged in the medium.

7. Plug the injection end of the endo-in delivery device with a thin piece of wet eye spear or sponge for security; endo-in delivery device is ready for transportation.

8. Place the endo-in delivery device in an upright position (injection end down) into the injector carrier, where the container includes corneal storage media.

Various embodiments of the injector carrier can be used (e.g., in an operating room) for administration of a prepared graft as loaded into the endo-in delivery device as follows.

### Assemble Endo-In Delivery Device in Operating Room

1. Prepare basin with an intraocular irrigating solution (e.g., balanced salt solution) enough to submerge a syringe and endo-in delivery device.

2. Gently remove endo-in delivery device containing ophthalmic tissue from the container of corneal storage media.

3. Place the endo-in delivery device into the prepared basin with balanced salt solution, decouple the tubing from the stem, leaving the tubing attached to the endo-in delivery device in the basin, making sure the endo-in delivery device is submerged and no air bubbles observed inside the endo-in delivery device.

4A. As submerged, slowly connect the tubing attached at an end of the endo-in delivery device to valve that is connected to a syringe with balanced salt solution and leave it in basin with balanced salt solution.

4B. As submerged, slowly connect the tubing attached at an end of the endo-in delivery device to a syringe with balanced salt solution and leave it in basin with balanced salt solution. That is, as an alternative to step 4A, use of the valve may be omitted in cases where the endo-in delivery device is configured for connection directly to the syringe.

5. As submerged, gently place the free end of the endo-in delivery device against the wall of the basin, so the sponge, plug, or spear (herein called a “plug” for reference) touches that wall. Very slowly depress the plunger of the syringe to transfer balanced salt solution from the syringe to the endo-in delivery device, ensuring there are no air bubbles present. Corneal storage medium will start exiting endo-in delivery device through the free end and plug and balanced salt solution will replace corneal storage medium inside of the endo-in delivery device.

6. Leave prepared syringe with connected endo-in delivery device in the basin.

7. Prepare recipient/patient.

8. As submerged, remove plug from the end of the endo-in delivery device.

9. Remove the prepared syringe from the basin with connected endo-in delivery device by holding syringe body and transfer onto operating field.

10. Place the injection end of the endo-in delivery device next to the periphery of the patient's cornea.

11. By pushing on the syringe body, insert one end of the endo-in delivery device into the anterior chamber of the recipient through a pre-cut incision or through an incision made using a cutting surface of the endo-in delivery device, and make sure the end is completely visible in the chamber.

12. Gently depress the plunger of the syringe to transfer prepared ophthalmic tissue graft from the endo-in delivery device into the anterior chamber of the recipient, ensuring there are no air bubbles present.

13. Confirm the presence of the graft in the cavity of the anterior chamber and gently remove the injection end of the endo-in delivery device from the anterior chamber.

14. The ophthalmic tissue graft should naturally unfold from the tri-fold position, but if needed, perform appropriate steps to unfold donor ophthalmic tissue graft within the eye.

Although the foregoing description provides embodiments of the invention by way of example, it is envisioned that other embodiments may perform similar functions and/or achieve similar results. Any and all such equivalent embodiments and examples are within the scope of the present invention.

What is claimed is:

1. A delivery apparatus for performing endothelial keratoplasty comprising:
(a) a first conduit having a beveled first end with a first opening;
(b) a second conduit having a second end with a second opening; and
(c) a chamber that extends between the first conduit and the second conduit along a first axis, wherein:
(i) the chamber has an interior thickness, the interior thickness extends along a second axis perpendicular to the first axis, and the interior thickness is between 0.4 millimeters and 0.8 millimeters, and wherein
(ii) the chamber has an interior width, the interior width extends along a third axis perpendicular to the first axis and the second axis, and the interior width is between 2 millimeters and 4 millimeters.

2. The delivery apparatus of claim 1, wherein:
(a) the first conduit comprises an inner diameter between 1.1 millimeters and 2.2 millimeters; and
(b) the second conduit comprises an inner diameter between 1.1 millimeters and 2.2 millimeters.

3. The delivery apparatus of claim 1 further comprising a pressure actuated valve, wherein the pressure actuated valve comprises:
(a) a first portion configured for coupling to a syringe;
(b) a valve main body;
(c) a deformable stopper disposed in the main body, and
(d) a second portion in fluid communication with the injector second end.

4. The delivery apparatus of claim 3 further comprising:
(a) an elongated resilient member having a channel extending from a first aperture to a second aperture, wherein
(i) the resilient member is coupled to the second portion of the pressure-actuated valve by extending the second portion partially through the resilient member first aperture, and wherein
(ii) the resilient member is coupled to the delivery apparatus second end by extending the second end partially through the resilient member second aperture.

5. The delivery apparatus of claim 4 further comprising a syringe filled with a balanced salt solution coupled to the first portion of the pressure actuated valve.

6. The delivery apparatus of claim 5 further comprising a bulb disposed on an outer surface of the second conduit.

7. The delivery apparatus in claim 1, wherein
(a) the chamber houses an ophthalmic tissue graft comprising an endothelium layer and a stromal layer, and the ophthalmic tissue graft is in a folded configuration with the endothelium layer facing inward and the stromal layer facing outward to contact a chamber inner surface; and
(b) the ophthalmic tissue graft endothelium layer is in contact with a corneal storage medium

8. A delivery apparatus for performing endothelial keratoplasty comprising:
(a) a first conduit having a beveled first end with a first opening;
(b) a second conduit having a second end with a second opening; and
(c) a chamber that extends between the first conduit and the second conduit, wherein
(i) the chamber comprises a flattened, hollow body that is wider than the first conduit and wider than the second conduit, and wherein
(ii) the chamber is sized to accommodate cornea tissue with an endothelial cell layer on a first side while the cornea tissue is in a trifolded configuration with the endothelial cell layer facing inward away from an interior surface of the chamber.

9. The delivery apparatus of claim 8, wherein the second opening is coupled to, and in fluid motion with, a fluid manipulation device.

10. The delivery apparatus of claim 9, further comprising a delivery device carrier, wherein the delivery device carrier comprises:
(a) a container having an opening;
(b) a cap configured to seal the opening of the container; and
(c) at least a portion of the delivery device is disposed within the container.

11. The delivery apparatus in claim 10, wherein the delivery device and the container are each at least partially filled with a corneal storage medium.

12. The delivery apparatus according to claim 8, wherein:
(a) the beveled first end comprises a leading portion and a trailing portion;
(b) the leading portion comprises a cutting surface for cutting and penetrating eye tissue; and
(c) the trailing portion comprises a non-cutting surface.

13. The delivery apparatus according to claim 8 further comprising a pressure actuated valve coupled to the second end.

14. The delivery apparatus according to claim 13 further comprising a syringe coupled to the pressure actuated value.

15. The delivery apparatus according to claim 13, wherein the pressure actuated value comprises:
(a) a first portion configured for coupling to a syringe;
(b) a valve main body;
(c) a deformable stopper disposed in the main body; and
(d) a second portion coupled to the second end of the second conduit.

16. The delivery apparatus according to claim 14, wherein:
(a) the pressure actuated value is coupled to the second end through a resilient member; and wherein
(b) the resilient member comprises a channel extending from a first aperture to a second aperture; and wherein:
(c) the resilient member is coupled to the second portion of the pressure-actuated valve by extending the second portion partially through the resilient member first aperture; and wherein
(d) the resilient member is coupled to the second end by extending the second end partially through the resilient member second aperture.

17. A method for storing ophthalmic tissue graft with a layer of endothelial cells facing inward comprising:
(a) providing the delivery apparatus of claim 10;
(b) loading the chamber with an ophthalmic tissue graft in a tri-folded configuration with the layer of endothelial cells folded inward, wherein the layer of endothelium cells contacts the corneal storage medium;
(c) orienting the endothelium-in ophthalmic tissue graft relative to the orientation of the beveled first end using the bevel-up, endo-down technique wherein the endothelium layer faces away from a sloping portion of the first end; and
(d) transporting the delivery device and carrier to a site for performing endothelial keratoplasty.

18. A method for performing endothelial keratoplasty for an eye of a patient comprising:
(a) providing a delivery apparatus according to claim 3;
(b) loading the delivery apparatus with a corneal storage medium;
(c) loading the chamber with an ophthalmic tissue graft, wherein
(i) the ophthalmic tissue graft comprises an endothelium layer and a stromal layer opposite the endothelium layer,
(ii) the ophthalmic tissue graft is folded with the endothelium layer facing inward and the stromal layer facing outward to contact the chamber interior surface;
(iii) the ophthalmic tissue graft is oriented within the chamber using the bevel-up, endo-down technique;
(d) coupling a syringe to the first portion of the pressure actuated valve, wherein the syringe comprises (i) a barrel containing a fluid, and (ii) a plunger partially housed within the barrel such that depressing the plunger applies pressure to the fluid;
(e) creating an incision in the eye of a patient, wherein the incision is less than the outer diameter of the first conduit;
(f) inserting the beveled first end into the eye of a patient through the incision, wherein
(i) the beveled first end occupies substantially the entire incision, and wherein
(ii) the bevel faces upward in a direction away from the interior of the patient's eye; and
(g) depressing the plunger to dispense the ophthalmic tissue graft from the chamber through the beveled first end into the eye of the patient with the endothelial cell layer facing inward to the interior of the patient's eye.

19. The method for performing endothelial keratoplasty of claim 18, wherein depressing the plunger causes the fluid to flow from the barrel, through the pressure actuated valve and the delivery device, and into the patient's eye to exert a positive pressure within the eye.

20. The method for performing endothelial keratoplasty of claim 19, wherein the fluid is a balanced salt solution.
